(19) United States
(12) Patent Application Publication
(10) Pub. No.: US 2025/0295398 A1
(43) Pub. Date: Sep. 25, 2025

(54) DEVICE ENABLING WIRE CANNULATION

(71) Applicant: Boston Scientific Scimed, Inc., Maple Grove, MN (US)

(72) Inventors: Lauren Koon, Mississauga (CA); Christian Balkovec, Cambridge (CA)

(21) Appl. No.: 19/086,927

(22) Filed: Mar. 21, 2025

Related U.S. Application Data
(60) Provisional application No. 63/568,974, filed on Mar. 22, 2024.

(51) Int. Cl. A61B 17/00

(52) U.S. Cl. CPC A61B 17/00234; A61B 2017/00243; A61B 2017/00292

(57) ABSTRACT

A system for deployment of a pre-loaded guidewire for access to a heart is disclosed. The system includes a device comprising an elongated body having a body length, a proximal end and a distal end portion terminating in a distal tip and a lumen extending through the elongated body and being dimensioned to slidingly receive the pre-loaded guidewire. The system further includes a guidewire control device positioned on the device, configured to physically communicate with the pre-loaded guidewire and advance the pre-loaded guidewire longitudinally within the lumen. The pre-loaded guidewire includes an untriggered state and a triggered state. In the triggered state, the pre-loaded guidewire extends longitudinally within the lumen towards the distal tip of the device.

### CROSS REFERENCE TO RELATED APPLICATIONS

This application claims priority to U.S. Provisional Patent Application No. 63/568,974 entitled “DEVICE ENABLING WIRE CANNULATION,” filed Mar. 22, 2024, which is incorporated herein by reference in its entirety.

### TECHNICAL FIELD

The present disclosure relates to medical systems and methods for accessing a tissue within a heart of a patient. More specifically, the present disclosure relates to medical systems and methods for deployment of a pre-loaded guidewire for access to the heart and subsequent tissues within the heart.

### BACKGROUND

Generally, in cardiac procedures, several devices (e.g., a transseptal needle, a dilator, a sheath, multiple wires, a catheter, therapy devices or a combination thereof) may be used by a user simultaneously or in succession to access the heart and subsequent tissues within the heart. The handling of multiple devices requires the user of the medical devices to access specific anatomy within the heart, maintain control of the devices, while also simultaneously maintaining the correct position of the devices relative to each other. The potential consequences of an inadvertent movement of a hand or a finger on these devices may include an accidental puncture of a tissue, a suboptimal transseptal puncture location which may increase procedure complexity, tamponade, an accidental aortic puncture, loss of transseptal access which would require performing a second puncture, among others.

In these circumstances, it may be difficult to simultaneously cannulate a new wire through one of the devices, for example through the dilator, into a newly accessed space. However, feeding a wire into the newly accessed space, for example after needle-based punctures, is highly beneficial and in many circumstances required, because it secures access to the space and acts as a rail for sheaths, dilators, and therapy devices. Against this background, there exists a continuing need in the industry to provide improved devices and methods for gaining access to and puncturing a tissue within a patient's heart. An object of the present invention is therefore to provide such an apparatus.

### SUMMARY

In Example 1, a system for deployment of a pre-loaded guidewire for access to a heart includes a device comprising an elongated body having a body length, a proximal end and a distal end portion terminating in a distal tip and a lumen extending through the elongated body and being dimensioned to slidingly receive the pre-loaded guidewire. The system also includes a guidewire control device positioned on the device, configured to physically communicate with the pre-loaded guidewire and advance the pre-loaded guidewire longitudinally within the lumen; wherein the pre-loaded guidewire includes an untriggered state and a triggered state, and wherein in the triggered state the pre-loaded guidewire extends longitudinally within the lumen towards the distal tip of the device.

Example 2 is the system of Example 1 wherein the device is a puncture wire, and wherein the pre-loaded guidewire cannulates the puncture wire through the lumen of the puncture wire.

Example 3 is the system of any of Examples 1-2 further comprising a dilator defining a dilator lumen and having a handle at a proximal end portion of the dilator, wherein the guidewire control device is positioned on the dilator handle, and wherein the puncture wire is advanced through the dilator lumen.

Example 4 is the system of Example 1 wherein the device is a dilator having a handle at the proximal end of the elongated body, and wherein the guidewire control device is positioned on the dilator handle.

Example 5 is the system of Example 1 wherein the guidewire control device includes a trigger feature, and wherein when the trigger feature is actuated the pre-loaded guidewire is in the triggered state.

Example 6 is the system of Example 5 wherein the trigger feature is a button positioned on the device.

Example 7 is the system of Example 1 wherein the guidewire control device is a position dial.

Example 8 is the system of Example 7 wherein the position dial includes a traction element, and wherein friction applied by the position dial to the pre-loaded guidewire advances the pre-loaded guidewire longitudinally within the lumen.

Example 9 is the system of Example 8 wherein the position dial is in physical communication with the pre-loaded guidewire within the lumen, and wherein as the position dial is rotated the guidewire traverses forward towards the distal tip of the device.

Example 10 is the system of Example 8 wherein the speed at which the position dial is rotated correlates with the speed at which the pre-loaded guidewire extends through the lumen.

Example 11 is the system of Example 8 wherein a user may control the forward and backward movement of the pre-loaded guidewire by the rotation of the position dial.

Example 12 is the system of Example 4 wherein the pre-loaded guidewire includes at least one or more grooves located on a body of the pre-loaded guidewire, wherein the guidewire control device is a knob having a top portion protruding from the dilator handle and a bottom portion positioned within the lumen, and wherein when the knob aligns with one of the at least one or more grooves on the pre-loaded guidewire, a user longitudinally advances the top portion of the knob across the dilator thereby advancing the pre-loaded guidewire longitudinally within the lumen.

Example 13 is the system of Example 1 wherein the guidewire control device is an accessory device.

Example 14 is the system of Example 13 wherein the accessory device locks into the device and is in physically communication with the pre-loaded guidewire, controlling the longitudinal movement of the pre-loaded guidewire within the lumen.

Example 15 is the system of Example 1 wherein the guidewire control device allows the advancement of the pre-loaded guidewire through the device with minimal hand movement.

In Example 16, a system for deployment of a pre-loaded guidewire for access to a heart includes a tubular device comprising an elongated body having a body length, a proximal end and a distal end portion terminating in a distal tip, a lumen extending through the elongated body and being dimensioned to slidingly receive the pre-loaded guidewire. The system also includes a guidewire control device positioned on the device, physically communicating with the pre-loaded guidewire and controlling the lateral movement of the pre-loaded guidewire within the lumen; wherein the pre-loaded guidewire includes an untriggered state and a triggered state, and wherein in the triggered state the pre-loaded guidewire extends longitudinally within the lumen towards the distal tip of the device.

Example 17 is the system of Example 16 wherein the device is a puncture wire, and wherein the pre-loaded guidewire cannulates the puncture wire through the lumen of the puncture wire.

Example 18 is the system of Example 17 further comprising a dilator defining a dilator lumen and having a handle at a proximal end portion of the dilator, wherein the guidewire control device is positioned on the dilator handle, and wherein the puncture wire is advanced through the dilator lumen.

Example 19 is the system of Example 16 wherein the device is a dilator having a handle at the proximal end of the elongated body, and wherein the guidewire control device is positioned on the dilator handle.

Example 20 is the system of Example 16 wherein the guidewire control device includes a trigger feature, and wherein when the trigger feature is actuated the pre-loaded guidewire is in the triggered state.

Example 21 is the system of Example 20 wherein the trigger feature is a button on the handle.

Example 22 is the system of Example 16 wherein the guidewire control device is a position dial on the handle.

Example 23 is the system of Example 22 wherein the position dial includes a traction element, and wherein friction applied by the position dial to the pre-loaded guidewire advances the pre-loaded guidewire longitudinally within the lumen.

Example 24 is the system of Example 22 wherein the position dial is in physical communication with the pre-loaded guidewire within the lumen, and wherein as the position dial is rotated the guidewire traverses forward towards the distal tip of the device.

Example 25 is the system of Example 22 wherein the speed at which the position dial is rotated correlates with the speed at which the pre-loaded guidewire travels through the lumen.

Example 26 is the system of Example 22 wherein a user may control the forward and backward movement of the pre-loaded guidewire by the rotation of the position dial.

Example 27 is the system of Example 16 wherein the guidewire control device allows the advancement of the pre-loaded guidewire through the device with minimal hand movement.

In Example 28, a device for deployment of a pre-loaded guidewire for access to a heart includes a device comprising an elongated body having a body length, a proximal end and a distal end portion terminating in a distal tip and a lumen extending through the elongated body and being dimensioned to slidingly receive the pre-loaded guidewire. The device also includes a guidewire control device positioned on the device, configured to physically communicate with the pre-loaded guidewire and advance the pre-loaded guidewire longitudinally within the lumen; wherein the pre-loaded guidewire includes an untriggered state and a triggered state; wherein in the triggered state the pre-loaded guidewire extends longitudinally within the lumen towards the distal tip of the device; and wherein the device is a puncture wire, and wherein the pre-loaded guidewire cannulates the puncture wire through the lumen of the puncture wire.

Example 29 is the device of Example 28 further comprising a dilator defining a dilator lumen and having a handle at a proximal end portion of the dilator, wherein the guidewire control device is positioned on the dilator handle, and wherein the puncture wire is advanced through the dilator lumen.

Example 30 is the device of Example 28 wherein the guidewire control device includes a trigger feature, and wherein when the trigger feature is actuated the pre-loaded guidewire is in the triggered state.

Example 31 is the device of Example 30 wherein the trigger feature is a button on the dilator handle.

Example 32 is the device of Example 28 wherein the pre-loaded guidewire includes at least one or more grooves located on a body of the pre-loaded guidewire, wherein the guidewire control device is a knob having a top portion protruding from the dilator handle and a bottom portion positioned within the lumen, and wherein when the knob aligns with one of the at least one or more grooves on the pre-loaded guidewire, a user longitudinally advances the top portion of the knob across the dilator thereby advancing the pre-loaded guidewire longitudinally within the lumen.

Example 33 is the device of Example 28 wherein the guidewire control device is an accessory device.

Example 34 is the device of Example 33 wherein the accessory device locks into the dilator handle and is in physically communication with the pre-loaded guidewire, controlling the longitudinal movement of the pre-loaded guidewire within the dilator lumen.

In Example 35, a method for deployment of a pre-loaded guidewire includes providing a device comprising an elongated body having a body length, a proximal end and a distal end portion terminating in a distal tip and a lumen extending through the elongated body and being dimensioned to slidingly receive the pre-loaded guidewire. The method also includes providing a guidewire control device positioned on the device, configured to physically communicate with the pre-loaded guidewire and advance the pre-loaded guidewire longitudinally within the lumen; wherein the pre-loaded guidewire includes an untriggered state and a triggered state, and wherein in the triggered state the guidewire control device is in physical communication with the pre-loaded guidewire and the pre-loaded guidewire extends longitudinally within the lumen towards the distal tip of the device.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A-1B are schematic illustrations of a medical procedure within a patient's heart for gaining access to the transseptal and epicardial space and for use in ablating heart tissue, according to embodiments of the present disclosure.

FIG. 2 is an illustration of a system for deployment of a pre-loaded guidewire within a dilator, according to embodiments of the present disclosure.

FIG. 3 is a schematic illustration of an example guidewire control device positioned on the dilator of FIG. 2 to control the advancement of the pre-loaded guidewire, according to embodiments of the present disclosure.

FIGS. 4A-4B illustrate an example guidewire control device positioned on the dilator of FIG. 2 to control the advancement of the pre-loaded guidewire, according to embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

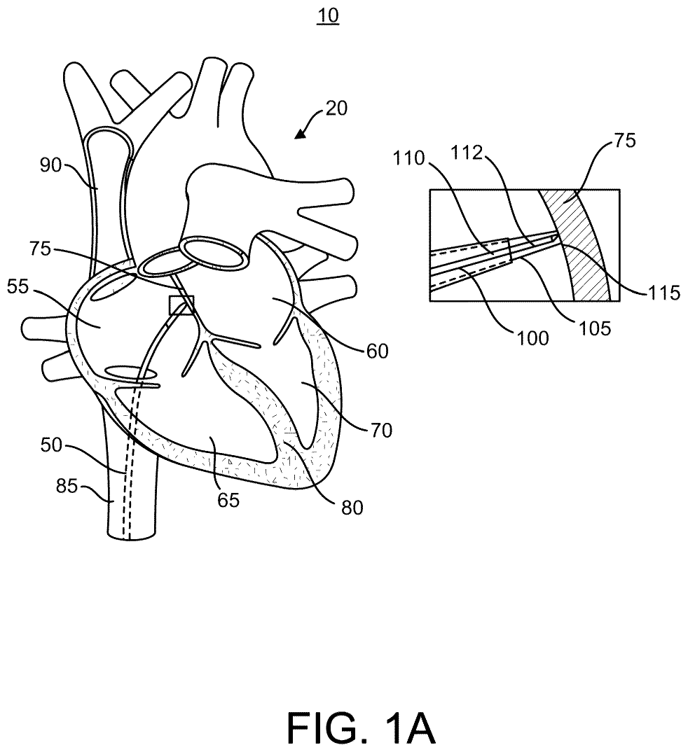

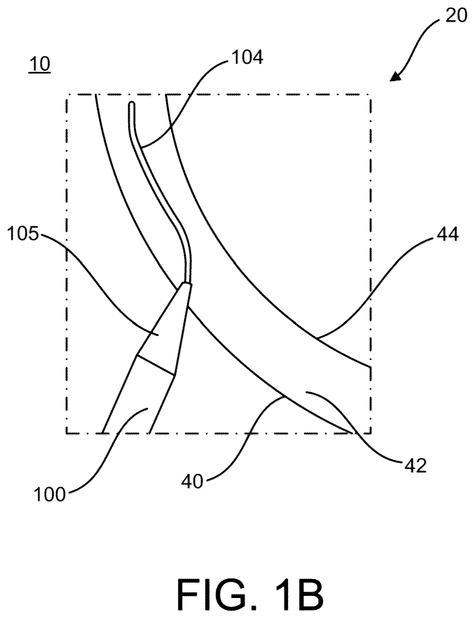

FIGS. 1A-1B are schematic illustrations of a medical procedure within a patient's heart for gaining access to the transseptal and epicardial space, according to embodiments of the present disclosure. FIG. 1A is an illustration of a medical procedure 10 within a patient's heart 20 utilizing a transseptal access system 50. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

Procedures for providing access to the left atrium 60 use transseptal access systems and devices for subsequent deployment of the aforementioned diagnostic and/or therapeutic devices within the left atrium 60. In these procedures, a target tissue site can be defined by tissue on the atrial septum 75. The target site is accessed via the inferior vena cava (IVC) 85, for example through the femoral vein, according to conventional catheterization techniques. In other embodiments, access to the target site on the atrial septum 75 may be accomplished using a superior approach wherein the transseptal access system 50 is advanced into the right atrium 55 via the superior vena cava (SVC) 90.

Transseptal access system procedures may include many devices like an introducer sheath 100, a dilator 105, a puncture device having distal end portion 112 terminating in a tip electrode 115, and a guidewire. In various embodiments, the puncture device 110 is a mechanical puncture device (e.g., a needle) or an RF perforation device. In an embodiment, the puncture device 110 can be disposed within the dilator 105, which itself can be disposed within the sheath 100. In another embodiment, the dilator 105 and the sheath 100 may be integrated into one device and the puncture device 110 may be disposed within the said device. In other embodiments, the dilator 105 and the puncture device 110 may be integrated into one device and then inserted into a sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator may be fully inserted into the sheath prior to entering the body, and both may be advanced simultaneously towards the heart. Alternatively, the dilator may then be introduced into the sheath and over the guidewire, and advanced through the sheath into the SVC.

When the guidewire, sheath 100 and dilator 105 have been positioned in the SVC 90, the guidewire is removed from the body, and the sheath 100 and the dilator 105 are retracted so that their distal ends are positioned in the right atrium 55. In an embodiment, the puncture device 110 described can then be introduced into the dilator 105, and advanced toward the heart 20. In another embodiment, the puncture device 110 may be introduced prior to retracting the sheath 100 and the dilator 105 from the SVC into the right atrium. In an embodiment, the puncture device 110 is then positioned such that the tip electrode 115 is aligned with or protruding slightly from the distal end of the dilator 105. In embodiments where the puncture device 110 is an RF perforation device, with the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 565 V (peak-to-peak), and functions to vaporize cells in the vicinity of the tip electrode, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds. In other embodiments, the step of delivering energy occurs over a period of about 300 milliseconds (ms).

Still another medical procedure 10 developed for diagnosing or treating physiological ailments originating within a heart 20 includes epicardial ablation to help restore a regular heart rhythm, as shown in FIG. 1B. As illustrated, the heart 20 includes a pericardium 40, a pericardial cavity 42 and a myocardium 44. The heart 20 is typically approached using a subxiphoid approach. Epicardial access is achieved via puncturing a layer of the pericardium 40 while avoiding the myocardium 44 of the heart. The pericardium 40 is a tough, double-walled, fibroelastic sac encompassing the heart 20 and the roots of the great vessels. The pericardium 40 includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium 40 and over the heart 20. This creates a pouch-like virtual or potential space around the heart 20 enclosed between the two opposing serosal surfaces, often referred to as the pericardial space or pericardial cavity 42.

In some embodiments, the pericardium 40 may be punctured with a puncture device 110, such as a needle (or other mechanical puncture device). Once punctured, a dilator 105 is advanced to dilate the puncture created by the needle through the pericardium 40. In certain embodiments, a sheath 100 may be advanced with the dilator 105 simultaneously. In other embodiments, the sheath 100 may be advanced afterwards. The sheath 100 and the dilator 105 may then be withdrawn to leave the guidewire 104 in the pericardial cavity 42. Minimally invasive access to the epicardium is required for diagnosis and treatment of a variety of arrhythmias and other conditions. During epicardial ablation, tiny scars are created on the outside of the heart to create a transmural lesion. In other words, to achieve an ablated tissue through the thick muscle of the heart.

The present disclosure describes novel systems and methods for providing safe access to the heart. As will be explained in greater detail herein, the embodiments of the present disclosure improve the means of complex cardiac procedures by providing a pre-loaded guidewire deployment mechanism.

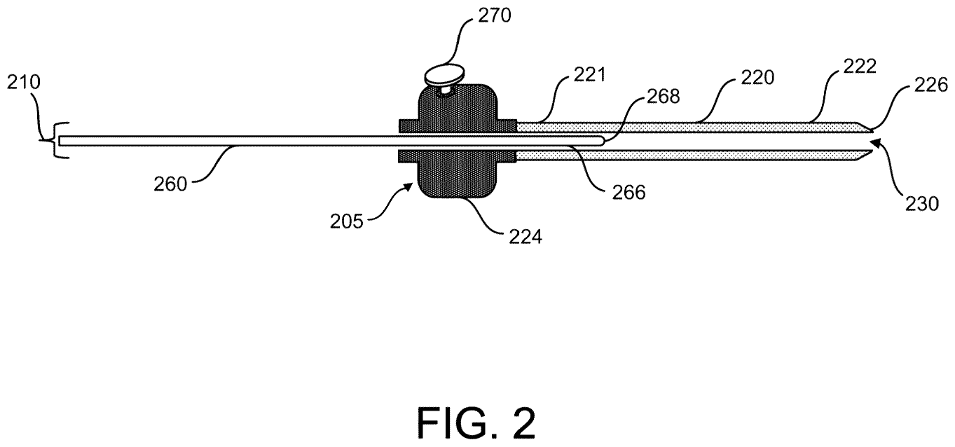

FIG. 2 is an illustration of a system for deployment of a pre-loaded guidewire 210 integrated with a dilator 205 for access to a heart, according to an embodiment of the present disclosure. As shown, the dilator 205 includes an elongated dilator body 220 having a body length, a dilator handle 224 having a handle length, and a dilator lumen 230 extending longitudinally through the handle 224 and the dilator body 220. Additionally, the dilator body 220 includes a proximal end portion 221 and an opposite distal end portion 222 terminating in a distal tip 226. In an embodiment, the handle 224 is connected to the proximal end portion 221 of the dilator body 220. In an embodiment, the body length and the handle length together define a dilator length. Additionally, as shown, the lumen 230 of the dilator 205 is dimensioned to slidingly receive the guidewire 210. In some embodiments, the guidewire 210 may be pre-loaded into the dilator 205 when a procedure requires the use of a guidewire.

As can be further seen from FIG. 2, the pre-loaded guidewire 210 includes a guidewire body having a proximal portion 260 and a distal portion 266 extending from the proximal portion 260 and terminating in a distal tip 268. As will be appreciated, in some embodiments, the length of the guidewire 210 is greater than the length of the dilator 205 so that part of the proximal portion 260 of the guidewire 210 extends proximally of the handle 224 when the distal portion 266, particularly the distal tip 268, extends distally of the dilator 205, thus allowing the proximal portion 260 to be manipulated by the user as needed. In certain embodiments, the pre-loaded guidewire 210 may be inserted into the dilator 205 and passed through another medical device, for example a transseptal needle.

In another embodiment, the system for deployment of a pre-loaded guidewire 210 may include a puncture wire (not shown) comprising an elongated body having a body length, a proximal end and a distal end portion terminating in a distal tip and a hollow lumen extending through the elongated body and being dimensioned to slidingly receive the pre-loaded guidewire 210. In an embodiment, the pre-loaded guidewire 210 may canulate the puncture wire through the hollow lumen of the puncture wire. In some embodiments, the puncture wire may then itself advanced through the dilator 205 through the dilator lumen 230. In an embodiment, following the puncture of a target tissue, as described in FIGS. 1A-1B above, with the hollow lumen of the puncture wire, the pre-loaded guidewire 210 may then immediately advance through the hollow lumen of the puncture wire via a guidewire control device 270 positioned on the dilator 205 to secure access of the puncture site created by the puncture wire. In one embodiment, the puncture wire is a mechanical puncture device (e.g., a needle). In another embodiment, the puncture wire is an RF perforation device.

As can be further seen from FIG. 2 and explained in greater detail below, the system further includes the guidewire control device 270 positioned on the dilator 205. In some embodiments, the guidewire control device 270 may be positioned on the dilator handle 224. In an embodiment, the guidewire control device 270 is configured to physically communicate with the pre-loaded guidewire 210 and advance the pre-loaded guidewire 210 longitudinally within the dilator lumen 230. In other embodiments in which the pre-loaded guidewire 210 is cannulated into the puncture wire that is itself integrated with the dilator 205, the guidewire control device 270 is configured to physically communicate with the pre-loaded guidewire 210 within the puncture wire and advance the pre-loaded guidewire 210 longitudinally within the puncture wire hollow lumen. Thus, in some embodiments, the guidewire control device 270 may include an upper portion that protrudes from the dilator 205 for user manipulability, and a lower portion that extends through the dilator handle 224 and into the dilator lumen 230, and in some instances through the puncture wire lumen, and is in physical communication with the pre-loaded guidewire 210. In certain embodiments, the pre-loaded guidewire 210 includes an untriggered state in which the guidewire 210 is positioned within the dilator lumen 230, as shown in FIG. 2. In an embodiment, in the untriggered state, the distal portion 266 of the pre-loaded guidewire 210 may be positioned in the dilator lumen 230 near the dilator handle 224 or near the proximal end 221 of the dilator body 220. In certain embodiments, the pre-loaded guidewire 210 further includes a triggered state in which because of the actuation of the guidewire control device 270, the pre-loaded guidewire 210 advances longitudinally within the dilator lumen 230 towards the distal tip 226 of the dilator 205, or distal to the distal tip 226 of the dilator 205. In certain embodiments, the guidewire control device 270 may be a lever, a wheel, a screw, a button or any other structure that may be positioned on and within the dilator 205 and be configured to physically communicate with the pre-loaded guidewire 210 to advance the pre-loaded guidewire 210 longitudinally within the dilator lumen 230, and in some instances through the puncture wire lumen.

In an embodiment, the guidewire control device 270 may include a trigger feature in which when the trigger feature is actuated the pre-loaded guidewire 210 is in the triggered state. In an embodiment, the trigger feature may be a button located on the dilator handle 224. In an embodiment, the trigger feature may be actuated by user manipulation (i.e., pressing a button, rotating a wheel or a lever, among others).

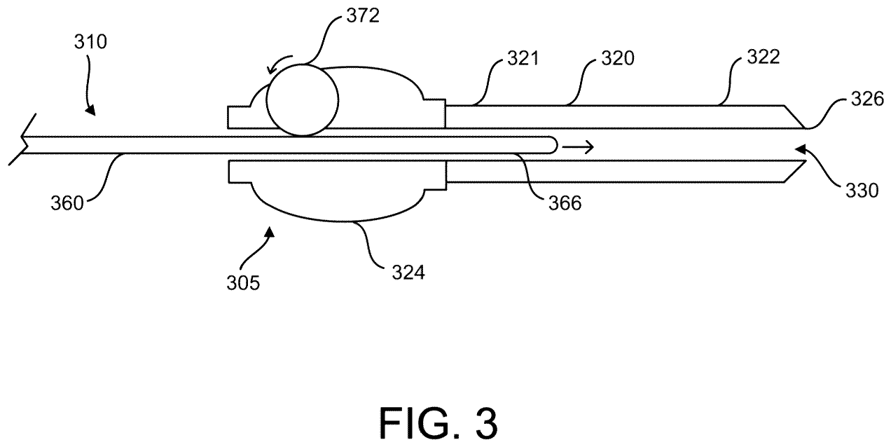

FIG. 3 is a schematic illustration of a position dial 372 positioned on a dilator 305 to control the advancement of a pre-loaded guidewire 310, according to an embodiment of the present disclosure. In an embodiment, the guidewire control device 270 of FIG. 2 may include a position dial 372 positioned on the dilator handle 324. As shown, the position dial 372 includes a top portion protruding from the dilator handle 324 for user manipulability, a middle portion positioned within the dilator handle 324, and a bottom portion that extends into the dilator lumen 330 and is in physical communication with the pre-loaded guidewire 310. In an embodiment, the position dial 372 may include a traction element, wherein friction applied by the position dial 372 to the pre-loaded guidewire 310 advances the guidewire 310 longitudinally within the dilator lumen 330, and in some instances through the puncture wire lumen. Accordingly, in an embodiment, as shown, when the user rotates the position dial 372, the traction element on the dial 372 causes the pre-loaded guidewire 310 to advance longitudinally within the dilator lumen 330, thus causing the pre-loaded guidewire 310 to be in the triggered state. In some embodiments, the user may rotate the position dial 372 with a finger (i.e., with minimal involvement of the palm and/or several fingers).

In an embodiment, as the user rotates the dial 372 counterclockwise, the guidewire 310 may advance towards the distal tip 326 of the dilator 305, as shown in FIG. 3. In other embodiments, as the user rotates the dial 372 clockwise, the guidewire 310 may advance towards the distal tip 326 of the dilator 305. In an embodiment, the speed at which the position dial 372 is rotated may correlate to the speed at which the guidewire 310 travels through the dilator lumen 330. In other embodiments, the user may rotate the position dial 372 in the opposite direction to retract the guidewire 310 into the dilator lumen 330 and into an untriggered state. In still other embodiments, the user may rotate the position dial 372 in the opposite direction to remove the pre-loaded guidewire 310 from the dilator 305 completely. Thus, in an embodiment, the user may control the forward and backward longitudinal movement of the pre-loaded guidewire 310 by the rotation of the position dial 372. In an embodiment, the position dial 372 may be made of polymer, metal, or a combination thereof. In certain embodiments, a feature may be added to a surface of the dilator lumen 330, opposite of the position dial 372, to reduce any friction damage to the guidewire 310 that may be caused by the traction element and rolling of the dial 372 onto the guidewire 310. In an embodiment, the feature may be included to a region opposite to the traction element. In another embodiment, the feature may be included on a section including but extending beyond the region opposite to the traction element, e.g., the whole lumen 330 or a part of the lumen 330. In certain embodiments, the feature may be a lubricating material, e.g., silicon, or may be a layer or coating, e.g., PTFE. In yet another embodiment, the feature may also include a moving element like a bearing, roller or wheel to facilitate translation of the guidewire.

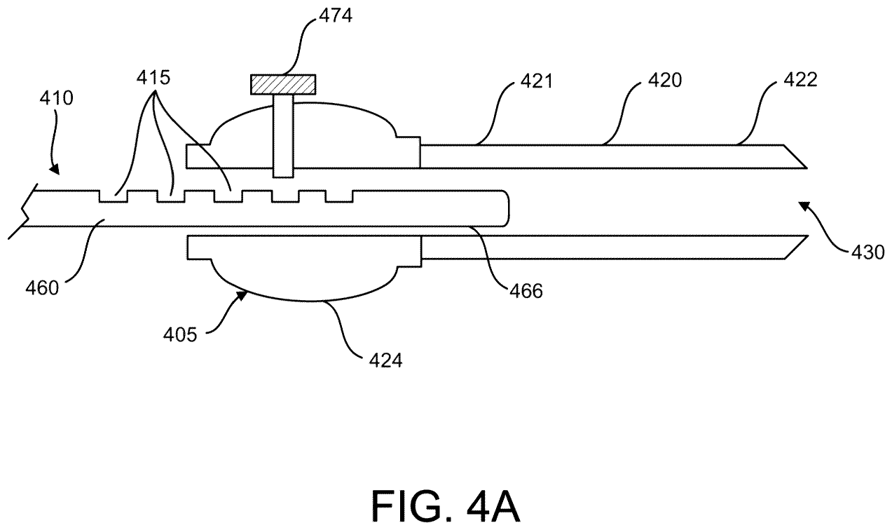

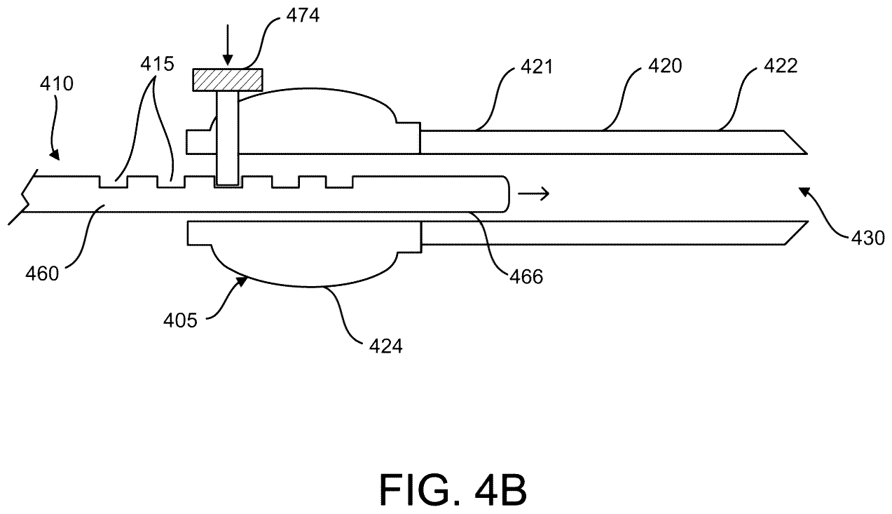

FIGS. 4A-4B illustrates a guidewire control device and a pre-loaded guidewire 410 having at least one or more grooves 415, according to an embodiment of the present disclosure. In an embodiment, the guidewire control device 270 positioned on the dilator 205 of FIG. 2 may include a knob 474 having a top portion protruding from the dilator handle 424 and a bottom portion positioned within the dilator lumen 430. In an embodiment, the pre-loaded guidewire 415 may include at least one or more grooves 415 located on the body of the pre-loaded guidewire 410. In an embodiment, the shape of the at least one or more grooves 415 may match the shape of the bottom portion of the knob 474 so that in a triggered state the bottom portion of the knob 474 may align with the at least one or more grooves 415 of the pre-loaded guidewire 410.

In certain embodiments, FIG. 4A depicts the untriggered state of the pre-loaded guidewire 410 in which the knob 474 is not actuated and is therefore not aligned with the at least one or more grooves 415 of the guidewire 410. In an embodiment, the knob 474 may be actuated by the user by pressing the knob 474 downwards while also slightly advancing the pre-loaded guidewire 474 within dilator lumen, if needed, to align the knob 474 with the at least one or more grooves 415 on the guidewire 410. In other embodiments, when the knob 474 aligns with one of the at least one or more grooves 415 on the pre-loaded guidewire 410, a user may longitudinally advance the top portion of the knob 474 across the dilator handle 424 thereby advancing the pre-loaded guidewire 410 longitudinally within the dilator lumen 430. In an embodiment, 1 mm movement of the knob 474 may translate to 1 mm movement of the guidewire 410. In another embodiment, 1 mm movement of the knob 474 may translate to 3 mm movement of the guidewire 410.

In certain embodiments, the guidewire control device 270 of FIG. 2 may be an accessory device that may connect to the dilator handle and be in physical communication with the pre-loaded guidewire. In an embodiment, the accessory device may control the longitudinal movement of the pre-loaded guidewire within the dilator lumen. In an embodiment, the guidewire control device allows the for the advancement of the guidewire through the dilator with minimal hand movement.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for deployment of a pre-loaded guidewire for access to a heart, the system comprising:
a tubular device comprising an elongated body having a body length, a proximal end and a distal end portion terminating in a distal tip, a lumen extending through the elongated body and being dimensioned to slidingly receive the pre-loaded guidewire; and
a guidewire control device positioned on the device, physically communicating with the pre-loaded guidewire and controlling the lateral movement of the pre-loaded guidewire within the lumen;
wherein the pre-loaded guidewire includes an untriggered state and a triggered state, and wherein in the triggered state the pre-loaded guidewire extends longitudinally within the lumen towards the distal tip of the device.

2. The system of claim 1, wherein the device is a puncture wire, and wherein the pre-loaded guidewire cannulates the puncture wire through the lumen of the puncture wire.

3. The system of claim 2, further comprising a dilator defining a dilator lumen and having a handle at a proximal end portion of the dilator, wherein the guidewire control device is positioned on the dilator handle, and wherein the puncture wire is advanced through the dilator lumen.

4. The system of claim 1, wherein the device is a dilator having a handle at the proximal end of the elongated body, and wherein the guidewire control device is positioned on the dilator handle.

5. The system of claim 1, wherein the guidewire control device includes a trigger feature, and wherein when the trigger feature is actuated the pre-loaded guidewire is in the triggered state.

6. The system of claim 5, wherein the trigger feature is a button on the handle.

7. The system of claim 1, wherein the guidewire control device is a position dial on the handle.

8. The system of claim 7, wherein the position dial includes a traction element, and wherein friction applied by the position dial to the pre-loaded guidewire advances the pre-loaded guidewire longitudinally within the lumen.

9. The system of claim 7, wherein the position dial is in physical communication with the pre-loaded guidewire within the lumen, and wherein as the position dial is rotated the guidewire traverses forward towards the distal tip of the device.

10. The system of claim 7, wherein the speed at which the position dial is rotated correlates with the speed at which the pre-loaded guidewire travels through the lumen.

11. The system of claim 7, wherein a user may control the forward and backward movement of the pre-loaded guidewire by the rotation of the position dial.

12. The system of claim 1, wherein the guidewire control device allows the advancement of the pre-loaded guidewire through the device with minimal hand movement.

13. A device for deployment of a pre-loaded guidewire for access to a heart, the device comprising:
a device comprising an elongated body having a body length, a proximal end and a distal end portion terminating in a distal tip and a lumen extending through the elongated body and being dimensioned to slidingly receive the pre-loaded guidewire; and
a guidewire control device positioned on the device, configured to physically communicate with the pre-loaded guidewire and advance the pre-loaded guidewire longitudinally within the lumen;
wherein the pre-loaded guidewire includes an untriggered state and a triggered state, and wherein in the triggered state the pre-loaded guidewire extends longitudinally within the lumen towards the distal tip of the device; and
wherein the device is a puncture wire, and wherein the pre-loaded guidewire cannulates the puncture wire through the lumen of the puncture wire.

14. The device of claim 13, further comprising a dilator defining a dilator lumen and having a handle at a proximal end portion of the dilator, wherein the guidewire control device is positioned on the dilator handle, and wherein the puncture wire is advanced through the dilator lumen.

15. The device of claim 13, wherein the guidewire control device includes a trigger feature, and wherein when the trigger feature is actuated the pre-loaded guidewire is in the triggered state.

16. The device of claim 15, wherein the trigger feature is a button on the dilator handle.

17. The device of claim 13, wherein:
the pre-loaded guidewire includes at least one or more grooves located on a body of the pre-loaded guidewire;
the guidewire control device is a knob having a top portion protruding from the dilator handle and a bottom portion positioned within the lumen; and
when the knob aligns with one of the at least one or more grooves on the pre-loaded guidewire, a user longitudinally advances the top portion of the knob across the dilator thereby advancing the pre-loaded guidewire longitudinally within the lumen.

18. The device of claim 13, wherein the guidewire control device is an accessory device.

19. The device of claim 18, wherein the accessory device locks into the dilator handle and is in physically communication with the pre-loaded guidewire, controlling the longitudinal movement of the pre-loaded guidewire within the dilator lumen.

20. A method for deployment of a pre-loaded guidewire, the method comprising:
providing a device comprising an elongated body having a body length, a proximal end and a distal end portion terminating in a distal tip and a lumen extending through the elongated body and being dimensioned to slidingly receive the pre-loaded guidewire; and
providing a guidewire control device positioned on the device, configured to physically communicate with the pre-loaded guidewire and advance the pre-loaded guidewire longitudinally within the lumen;
wherein the pre-loaded guidewire includes an untriggered state and a triggered state, and wherein in the triggered state the guidewire control device is in physical communication with the pre-loaded guidewire and the pre-loaded guidewire extends longitudinally within the lumen towards the distal tip of the device.
